(19) United States
(12) Patent Application Publication
(10) Pub. No.: US 2023/0329832 A1
(43) Pub. Date: Oct. 19, 2023

(54) A Breast Tissue Marker and Localization System

(71) Applicant: CLEMSON UNIVERSITY, Clemson, SC (US)

(72) Inventors: Scott R. SLANEY, Clemson, SC (US); Delphine DEAN, Clemson, SC (US)

(21) Appl. No.: 17/772,803

(22) PCT Filed: Oct. 21, 2020
(86) PCT No.: PCT/US2020/056637

§ 371 Date: Apr. 28, 2022

Related U.S. Application Data
(60) Provisional application No. 62/924,928, filed on Oct. 23, 2019.

(51) Int. Cl. A61B 90/00; A61B 34/20; A61L 31/18; A61L 31/12

(52) U.S. Cl. CPC A61B 90/39; A61B 34/20; A61L 31/18; A61L 31/124; A61B 2090/3908; A61B 2090/3954; A61L 2400/12; A61B 2034/2051

(57) ABSTRACT

Provided is an improved breast tissue marker and localization system and method of use. The breast tissue marker and localization system comprising a biocompatible marker and a detector. The biocompatible markers comprise a polymeric binder and a magnetically susceptible material such as an iron oxide nanoparticle wherein the biocompatible marker is suitable for implantation for localization of an area of pathological interest. The detector is capable of detecting a response magnetic field in said biocompatible marker.

### CROSS-REFERENCE TO RELATED APPLICATIONS

This application claims priority to U.S. Provisional Application No. 62/924,928 filed Oct. 23, 2019 which is incorporated herein by reference.

### FIELD OF THE INVENTION

The present invention is related to an improved breast tissue marker and localization system for use in diagnostic breast evaluation.

### BACKGROUND

Each year, millions of women with suspicious masses and calcifications of the breast will undergo investigational core needle biopsies to determine the presence or absence of cancerous lesions in the breast. Once a potentially pathological region is identified, a patient undergoes an investigational breast biopsy in order to determine whether it is cancerous. Depending on patient details and physician preference, one of several biopsy methods may be performed to remove a very small sample of tissue from the suspect mass. The least invasive option is fine needle aspiration (FNA) in which a small gauge hollow needle is inserted into the target region and fluid tissue is removed via syringe. While this method results in the removal of minimal mass, it is possible for the sampled area to be so small that it does not incorporate any cancer cells which may result in a false positive test. Alternatively, a core needle biopsy (CNB) may be performed with a larger gauge needle which cuts and retracts a small cylinder of tissue which is then removed through the lumen of the needle. Oftentimes, CNB allows for multiple tissue cores to be harvested and removed while the needle remains in place. Physicians commonly require image guidance, either by ultrasound, MRI, or mammogram, to locate an area of interest.

Concurrent with this diagnostic biopsy, a small tissue marker known as a “clip” is typically delivered to the site via a spring-loaded injector. Clips; which typically comprise titanium, stainless steel, or nitinol; mark the biopsy location or an area of pathological interest. If it becomes necessary for a physician to revisit the biopsy site, implanted clips serve as a critical reference and aid in re-localization of the suspect mass.

If a lumpectomy, also referred to as a sentinel lymph node biopsy or SLNB, is indicated it is essential that the operating physician be able to accurately define the region of tissue to be excised. Failure to do so could result in positive margins, wherein the cancer cells are found out to the edge of the resected tissue. This would indicate that the cancerous tissue was incompletely removed. A surgeon's objective is to achieve negative margins, wherein a rim of healthy cells encircles the resected mass, while minimizing the amount of healthy tissue removed. Thus, proper localization of the target biopsy site is vital to the completion of a successful lumpectomy. In order to re-locate the biopsy site, a physician utilizes the previously implanted tissue marker. However, due to their size and composition, finding the implanted breast marker itself requires its own preoperative localization procedure. This typically involves the image-directed insertion of a long stainless-steel guidewire into the patient's breast in a radiology suite. The patient is then taken to the operating room for surgery.

The current standard of care for finding breast clips is wire-localization (WL). WL is an invasive and painful preoperative procedure that involves the insertion of a stainless-steel wire into the breast tissue under image guidance. Not only is the procedure regularly cited as traumatic for patients, it also places strain on medical personnel and leads to inefficiency in the use of hospital resources, imaging facilities, and medical personnel.

Various alternative methods have been introduced which provide day-of intraoperative localization through the placement of a specialized companion marker and a detection probe. However, the specialized companion markers are only temporary and must be placed next to existing diagnostic biopsy markers. Preoperative localization is thus still necessary in order to properly place them in the days preceding surgery. Though an improvement upon wire-localization, these systems have achieved minimal degrees of clinical acceptance.

Provided herein is a novel method of biopsy marker localization. The intraoperative detection system could reduce or eliminate the need for preoperative wire-localization thereby reducing hospital expenses, mitigating inefficiencies, improving patient outcomes, and expanding the patient population capable of benefitting from lumpectomy as a surgical option.

### SUMMARY OF THE INVENTION

The present invention is related to an improved breast tissue marker and localization system for localization of an area of pathological interest.

A particular feature of the invention is the ability to utilize a breast tissue marker which is bio-compatible and which allows for accurate localization of an area of pathological interest without the use of wire localization techniques.

These and other embodiments, as will be realized, are provided in a breast tissue marker and localization system comprising a biocompatible marker and a detector. The biocompatible markers comprise a polymeric binder and a magnetically susceptible material such as an iron oxide nanoparticle wherein the biocompatible marker is suitable for implantation for localization of an area of pathological interest. The detector is capable of detecting a response magnetic field in said biocompatible marker.

Yet another embodiment is provided in a method for localizing an area of pathological interest comprising:

placing a biocompatible marker in a position relative to the area of pathological interest wherein the biocompatible marker comprises a polymeric binder and a magnetically susceptible material such as an iron oxide nanoparticle wherein the biocompatible marker is suitable for implantation for localization of an area of pathological interest; and
detecting the biocompatible marker by a detector capable of detecting a response magnetic field in the biocompatible marker.

### BRIEF DESCRIPTION OF FIGURES

FIG. 1 is a schematic representation of procedural workflow using the inventive system and novel biopsy marker.

FIG. 2 is rendered drawings of representative coils and probe housing.

FIG. 3 illustrates contour plots of magnetic field strength in relation to a simulated retractor both with and without shielding.

FIG. 4 is a graphical illustration showing signal strength increasing as proximity to probe decreases.

FIG. 5 is a graphical illustration showing the distribution of signal responses to C4 at each distance among 10 trial

### DESCRIPTION

The present invention is related to an improved breast tissue marker and localization system. The marker comprises magnetically susceptible nanoparticles embedded in a polymeric carrier. The marker can be located by magnetism and allows for indications of distance from the marker with auditory and graphical displays of distance.

The inventive detector system enables the intraoperative localization of implanted breast biopsy markers, or markers indicating areas of pathological interest, by providing continuous distance-to-marker feedback preferably through at least one of a graphical display or auditory tones. The system operates on the physical principal of magnetism, emitting magnetic waves at a relatively low frequency which pass into an implanted clip. A current is induced in the clip which thus produces its own, smaller magnetic field preferably as a response magnetic field. The response magnetic field is detected by a highly sensitive handheld probe which captures field perturbances as deviations in the system's DC voltage. The degree of voltage change from the baseline attenuates with distance and is translated into graphical and auditory representations which aid the user in identifying the direction and distance of maximum signal. Critically, this detection unit is paired with a detectable clip that is suitable for long-term implantation. A probe capable of detecting a primary diagnostic biopsy marker facilitates true intraoperative localization of areas of pathological interest by eliminating the need for secondary placement in the days or weeks preceding surgery. Rather, the detectable clip or marker itself may serve as the primary, preferably only, biopsy site indicator effectively consolidating localization and allowing complete decoupling of marker placement and excision.

More specifically, the present invention is related to the development, fabrication, and assessment of a novel detection system that enables true intraoperative localization of primary diagnostic breast markers. The first element of the system is a handheld detector that functions on the principals of magnetism to guide a surgeon's incisional path by providing continuous graphical and audio feedback. In an embodiment of the invention the detector is capable of reading the presence of a nitinol marker as far as 4.5 cm away and that signal strength attenuates with distance.

An additional element of the detection system is a novel tissue marker which compliments the detection probe's operating principals. A composite of polymer with embedded metallic nanoparticles provides biocompatible and magnetic susceptible marker capable of serving as a primary diagnostic breast biopsy marker that confers intraoperative detectability. In one embodiment detection proximity is reported through a graphical user interface (GUI) capable of evaluating signal strength among various marker compositions. The real-time GUI is an effective means of acquiring data and visualizing feedback. The marker is safe and suitable for long-term implantation.

The inventive detection system is capable of detecting biopsy markers at clinically relevant distances and that the companion marker designed to complement this task could be an effective diagnostic breast biopsy marker. The combination of these technologies sets the foundation for a comprehensive intraoperative localization system which could reduce both patient suffering and clinical inefficiency by serving as a viable alternative to wire-guided localization.

The tissue marker comprises a composite of poly(lactic-co-glycolic acid) (PLGA) and iron oxide nanoparticles (IOPNs). The IOPNs confer magnetically susceptibility while remaining biocompatible and biodegradable thereby providing optimum detectability and clinical utility.

The marker degrades along a highly tailorable profile thereby allowing the polymeric binder to release IONPs at a biologically tolerable rate. Though initially contemplated for use in breast tissue the technology may be applied to other commercial markers so that they may become suitable for magnetic detection.

The continuous phase of the biopsy marker, in a preferred embodiment, comprises a biocompatible polymer, such as PLGA or polylactic acid (PLA). These polymers are well characterized and commonly used in FDA approved implants. Their constituents, PLA and PGA, degrade by hydrolysis into lactic acid and glycolic acid which are readily eliminated through metabolic pathways. These polymers may be tailored to degrade anywhere along a continuum of days to years depending on manufacturing methods that control crystallinity and, in the case of PLGA, the ratio of PLA to PGA. While 50:50 PLGA was selected for the fabrication of prototype markers, alternative polymeric carrier compositions could be utilized to modify degradation behavior.

The composite marker owes its magnetic susceptibility to a magnetically susceptible material which is preferably an iron oxide and most preferably iron (III) oxide (Fe₂O₃) nanoparticles (IONP) embedded within the polymer. These particles are the oxidized variety of Fe₃O₄ nanoparticles, both of which are utilized in a growing variety of biomedical applications. Through their incorporation into the polymeric carrier, IONPs provide a source of magnetically susceptible material. Whereas the equivalent mass of bulk iron would not be suitable for implantation due to toxicity, nanoparticles provide a means of delivering iron-containing material that is not present in high enough concentration to elicit an adverse cellular response.

As they are degraded, iron oxide nanoparticles are reduced into free Fe ions. An accumulation of these ions can lead to a homeostatic imbalance that results in cytotoxicity, generation of reactive oxygen species (ROS), inflammation, and even DNA damage. Owing to their exceptionally small size, IONPs have a very high surface area to volume ratio which enhances their reactivity, particularly in the case of ROS production. As free Fe ions migrate across the mitochondrial membrane, they react with hydrogen peroxide and produce highly reactive hydroxyl radicals which contribute to cellular and DNA damage. Given this mechanism, there is some indication that Fe₂O₃, the particle chosen in the prototype marker formulation, may be more stable than its counterpart, Fe₃O₄. This is because Fe₃O₄ has a greater oxidation potential.

In principle, iron oxide nanoparticles are agreeable to in vivo environments as they can be cleared from circulation by endogenous iron hemostatic pathways. This is partly because the body's handling of IONPs is largely dependent on the particles' particular physicochemical characteristics, including hydrodynamic size, oxidation state, and surface chemistry. A particle's hydrodynamic size is determined by the culmination of the core particle, any polymeric coating addition, and the corona of proteins accumulated during circulation. IONP with average particle sizes of 1 nm to 150 nm is preferred. More preferably, the IONP has an average particle size of 1 to 10 nm. An IONP greater than 100 nm in diameter rapidly opsonize, absorbing plasma proteins until a corona is formed. The presence of a protein corona initiates macrophage recognition, leading to rapid phagocytosis and subsequent sequestering in the liver and spleen. Particles smaller than 50 nm opsonize more slowly and thus take longer to enter into the reticuloendothelial system before accumulation in the liver and spleen. Once in the liver or spleen, nanoparticles are degraded and either excreted or reduced into iron ions. These ions are shuttled by ferritin and transferrin and are ultimately incorporated into new hemoglobin. Very small nanoparticles, 10 nm or smaller, are thought to be easily disposed of through renal clearance, a preferred pathway that limits iron retention. Several FDA cleared IONP-based products, including Feridex® and Feraheme® are already in use and can be delivered in doses exceeding 1 g and preferably 1-10 g. However, the potential for cytotoxicity remains and is considered a function of concentration. At lower concentrations, clearance pathways are able to keep pace and dispose of the particles, while higher concentrations may overwhelm clearance mechanisms and lead to cellular damage. It is preferable that the marker does not allow a blood level of IONP to exceed 200 μg/mL and preferably not 50 μg/mL.

Surface treatments are commonly applied to IONPs to confer improved biocompatibility or to act as an intermediary onto which a ligand cargo can bind. These are referred to in the art as core-shell nanosystems, and any number of shell compositions can be implemented depending on intended use. Most popular are a variety of polymers preferably selected from the group consisting of polyethylene glycol (PEG), poly(vinylpyrrolidone) (PVP), poly(vinyl alcohol) (PVA), PLGA, and chitosan. Silica (SiO₂), an inorganic compound, or dextran, an organic compound, are also frequently used in lieu of polymeric options. These coating materials are designed to promote biocompatibility by limiting oxidation and provide stability by preventing aggregation.

This work has produced a detection system for the localization of breast tissue markers utilizing the principles of magnetism. Verification and validation testing demonstrated that the prototype detector is capable of detecting breast biopsy markers at clinically relevant distances through breast tissue. A companion PLGA-IONP composite marker was also developed which complements the operating principles of the detection system. This novel marker was validated for use in conjunction with the detection system. A proximity study demonstrated clinically relevant detectability through the use of an end-user feedback interface.

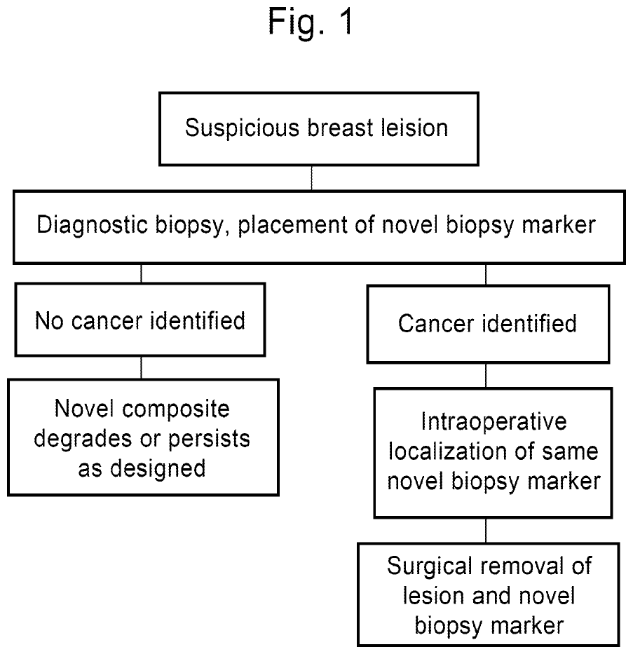

An ideal, clinically useful marker must be compatible with the described detection system and enable user-visualized localization at a minimum of 10 cm, more preferably 5 cm, even more preferably 4.5 cm and preferably a minimum of 3.0 cm. Such a marker and detection system could significantly improve the procedural workflow preceding surgery illustrated graphically in FIG. 1 wherein provided is a schematic of procedural workflow using the inventive system. It is also possible that the novel composite formulation could be applied to existing commercial metallic markers. This could be useful when it is preferred that a biopsy site remain identified indefinitely. A metallic marker embedded within a magnetically-enhanced composite would confer compatibility with a metal detection-based localization system for the duration of the composite, providing a period of time to allow for surgical intervention if necessary. Once the composite degrades and only the metallic marker remains, the patient may receive ongoing surveillance and diagnostic MRI imaging undistorted by highly susceptible magnetic artifacts.

The composite biopsy marker enables long term magnetic localization without introducing significant cytotoxicity. While PLGA forms the continuous phase of the marker, iron oxide nanoparticles embedded within the PLGA provides a source of magnetic susceptibility. A series of formulations were fabricated and formed into compact discs, each with a different ratio of PLGA:IONP.

Though PLGA offers excellent biocompatibility, manufacturability, and design specificity, embedded IONPs may not release in a biocompatible fashion. While not limited to theory, it is postulated that the first stage of PLGA degradation is marked by a decrease in polymer chain length, rather than by surface erosion. The result is that the bulk component loses weight but maintains its overall geometry. As the density of the polymer carrier decreases, its porosity increases. It is possible that this degradation behavior could cause the IONPs to undergo bulk release. Such a rapid release could compromise the intended effect of the carrier, allowing a high concentration of IONP to be released at potentially toxic levels.

In one embodiment, a coating material is added to the nanoparticle component in an effort to improve biocompatibility. The polymeric coating would have to withstand the processing conditions imposed during both solvent casting and compression molding. The coating is preferably insoluble in the solution and capable of dissolving the carrier and have a glass transition temperature above that of the carrier. In one embodiment a solution is prepared in a non-polymeric coating such as chitosan or dextran.

In one embodiment, PLA could be employed as or with a carrier polymer. Given its slow degradation, this carrier material could mitigate the effects of burst release. Alternatively, it may be possible to add an iron chelator to the polymeric carrier composition to assist degradation.

Some biopsy markers employ anti-migration techniques to reduce displacement of the clip from the intended site. In one embodiment, an adhesive surface chemistry is integrated into the exterior of the polymeric carrier. Conjugating a matrix binding protein, such as fibronectin, to the carrier surface could facilitate nonspecific binding with collagen surrounding the biopsy site thereby reducing marker migration.

Delivery of the composite marker into the biopsy site may be improved by altering its initial consistency. Rather than ejection of a bulk marker, it is possible that the composition could be delivered by injection as an aqueous or gel-like solution that cross-links in situ to form a solid implant. Once administered, the formulation would form to the surrounding tissue architecture, adopting a nonuniform geometry that could assist in migration mitigation. This delivery mechanism could allow a greater concentration of IONP to be incorporated into a smaller volume given that the polymeric carrier need not bind the implant into a rigid structure. Such an embodiment could be delivered through a small gauge needle such as a syringe, thereby increasing convenience and reducing patient discomfort.

Markers, also referred to as clips, are preferably delivered through an injector to the tumor bed within a breast. Placement of one or more markers typically occurs during the course of a primary breast biopsy to provide traceability in the event that subsequent care is required. The use of multiple markers, possibly of unique geometry, may be useful when patients present more than one lesion in the same breast. The purpose of multiple markers in such a setting is to provide discrimination between the various sites, which may differ in malignancy. A thorough assessment of distribution, extent, and malignancy of the disease allows surgeons to practice greater precision during surgical planning and work to reduce the volume of healthy tissue resected.

The inventive localization system utilizes the physical principal of magnetism to facilitate detection of implanted breast clips. Passing current down the length of a probe-like wand and into an inductor coil produces a magnetic field in accordance with Ampere's Law which projects into the breast tissue. Magnetic field strength (B) at a distance (D) from the edge of a solenoid is given by Equation 1:

B=μₒIn/2[(D+L)/√((D+L)\hat{ }2+R\hat{ }2)−D/√(D\hat{ }2+R\hat{ }2)]

where:
B=magnetic field strength;
D=distance from solenoid edge;
μₒ=magnetic permittivity of core;
I=current;
n=number of turns;
L=length; and
R=Radius.

When this field interacts with an implanted metallic clip or marker, a small current is induced in the clip. This produces a response magnetic field that is detected in the probe.

Current is drawn from a voltage regulator through a, preferably 15 pH, receiver coil and passed through the collector of a transistor and into a, preferably 1.8 mH, transmitter coil. The two coils share a common ferrite core, through which they are coupled. The placement of a capacitor in parallel with the transmit coil creates an LC tank circuit which resonates to produce a sinusoidal waveform, preferably of approximately 35 kHz for the purposes of demonstration of the invention. The sinusoidal waveform can range from about 30 kHz to 100 kHz or higher for demonstration of the invention. Current passing through the solenoid generates a magnetic field. The coupling that exists between the transmit and receive coils allows this waveform to cycle back through the transistor, forming an oscillator. This variable resistor which provides further DC bias refinement. After smoothening the oscillation into an essentially DC voltage, the signal is fed into a comparator along with 2.5V established through a voltage divider. When the DC bias is properly tuned, the signal voltage is also 2.5V and the comparator does not trigger.

However, if the magnetic field generated by the transmit coil propagates outward and interacts with a metallic marker, it induces small eddie currents which in turn generate a weak magnetic field of their own. Per the right-hand rule, this induced field is in the same direction of the transmit field, resulting in constructive interference. This causes a slight increase in size of the magnetic field surrounding the probe. Current is thus drawn to supply the expanding magnetic field. Due to Ohm's Law, a corresponding drop in voltage occurs which is fed into the comparator as a raw signal.

When the voltage drop exceeds a set threshold value, such as 30 mV, the comparator triggers, “swinging” the voltage to 5V. Because the magnitude of the response magnetic field scales linearly with the induced current, analyzing the attenuation of the response signal allows insight into the relative distance between a clip and the end of the wand. However, the effect of the comparator is akin to a non-linear amplifier. Care is taken to read the signal voltage returned by the component as it transitions from baseline to a differential voltage, such as 5V, in order to associate distance with voltage. An instrumentation amplifier is suitable to accentuate these voltage differences.

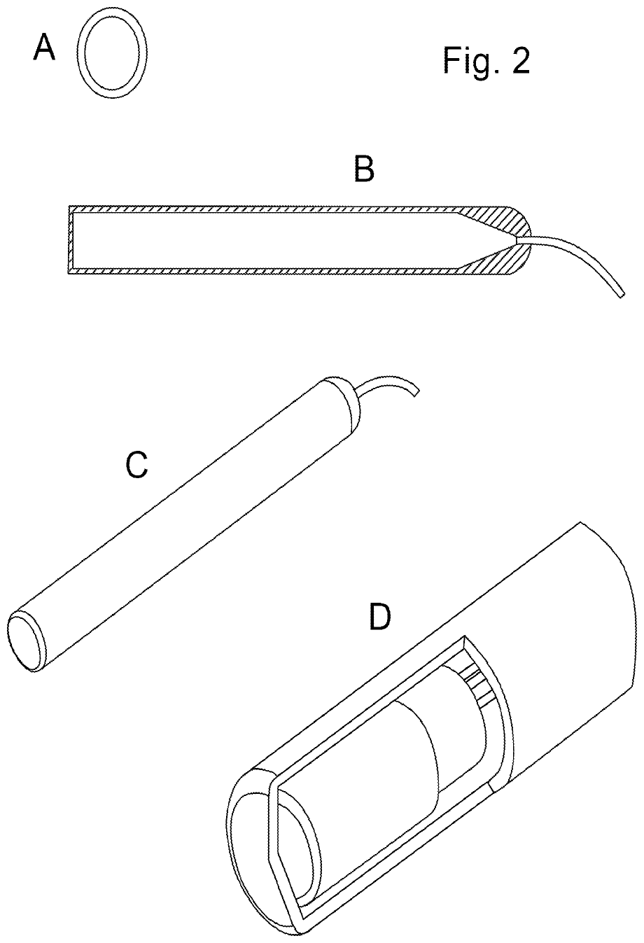

An embodiment of the detection device has inductor coils which are wound within the tip of a, preferably 15 cm, probe, preferably approximately 1.5 cm in diameter, is illustrated in FIG. 2. A lead connected to the handheld unit supplies power and reads the signal. The combination power/signal cable is fed to a distant data acquisition (DAQ-USB6002) component which is powered and relays the signal to a computer for generating a graphic and audio feedback interface. The cross-sectional shape of the detection device is preferably elongated and oblong, as illustrated in FIGS. 2A and 2B with a preferably flat operative tip as illustrated in FIG. 2C wherein the operative tip engages with the patient. The receiving coil is disposed towards the operative tip with wires extending opposite thereto as illustrated in FIG. 2D.

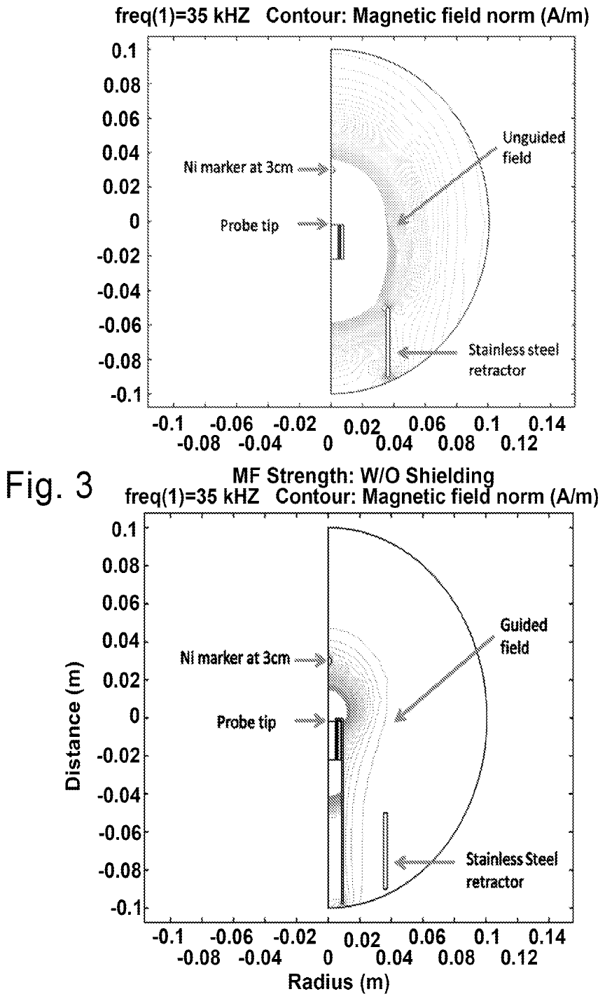

In a particularly preferred embodiment, a shielding device is included to prevent the errant detection of other metallic objects, such as surgical retractors, that may be present during surgery. An exemplary modeled embodiment of a 2D axisymmetric detector is illustrated in FIG. 3 based on COMSOL Multiphysics® software. A finite element analysis simulated magnetic field strength in a 10 cm radius around the distal end of the probe tip is illustrated in FIG. 3 wherein contour plots of magnetic field strength without, and with, shielding are illustrated graphically.

The simulations show half of the probe tip, an iron core and winding layers, and the generated magnetic field in two dimensions. The thin boxes in front of the probe tip and along the lateral side of the probe shows the addition of mu metal as a shielding material. A mu metal is a nickel-iron soft ferrous ferromagnetic alloy with high permeability which is well known in the art and widely available commercially. In the model, this is understood to be a two-dimensional representation of a revolution, thus making this shielding a cylinder revolved around the outside of the probe tip with a small opening on the end. A stainless-steel member added near the rear periphery of the probe represents retractors that are present during the surgery. The figures represent the magnetic field as isolines, along which all points have equal magnetic field strengths.

As illustrated in FIG. 3 the shielding strategy significantly reduces the strength of the magnetic field extending laterally from the probe while still allowing the field to propagate along the long axis toward the marker. This is essential for improving overall directionality such that only objects beyond the plane of the probe tip are detected due, in part, to the decrease in magnetic field at the retractor. It is advantageous to distinguish directional specificity from angular specificity. Angular specificity enables a user to infer the direction of a distant object by referencing the orientation of the probe tip whereas directional specificity provides boundary conditions which prohibit the field from extending into regions lateral to or behind the probe. Any detection ambiguity due to angular uncertainty is easily overcome as the clinician maneuvers the probe during the incision path. As the marker passes in and out of the field at different angles and depths, the focal signal origin becomes evident.

As a comparative example the detection of commercial markers indicated that it is possible to directly localize non-ferrous metallic markers. Among the markers assessed, the Tumark Vision® produced the most identifiable return signal, with early studies recording a maximum detection distance of approximately 4.5 cm. Though sufficient detection depth was achieved, the system suffered from noise comparable in amplitude to the signal, making reliable detection challenging.

Medical devices that produce a radio frequency electromagnetic field can transmit energy into the tissue of patients and practitioners. Specific absorbed radiation (SAR) is a value used to quantify, in units of W/kg, the power absorbed per mass of human tissue. The International Electrotechnical Commission (IEC) publishes SAR output standards for various medical technologies. A finite element model of the probe was analyzed in COMSOL Multiphysics to ensure compliance with IEC 60601-2-33-2010 which limits the SAR dose delivered to the trunk by a local transmit coil at 10 W/kg. To insure SAR is not exceeded a 10 g sample given properties attributable to breast adipose was placed directly adjacent to the distal tip of a 2D axisymmetric model detector probe. A maximum SAR value of 1.24×10⁻¹⁵ W/kg was recorded, far below the IEC regulatory guidelines.

Continuous user feedback is an advantageous embodiment of the invention which enables data collected by the probe to be translated into meaningful user feedback. An embodiment of a graphical user interface (GUI) was developed in LabVIEW. In one embodiment the GUI employs a series of incrementally activated indicator lights to communicate relative distance-to-clip by optical signal. Audio feedback in the form of increasing pitch from 300 to 2000 Hz is preferably supplied according to the same attenuation curve thereby communicating distance-to-clip by an audible signal. In order to properly calibrate the scale, a user must first initiate a baseline reading. In one embodiment the GUI will utilize distance vs signal data to provide a numeric distance estimation. Controls are provided to selectively enable audio and save collected data for further analysis. The indicators supplied in the user-facing control panel can be modified to accommodate a surgeon's preferred graphical and audio feedback preferences.

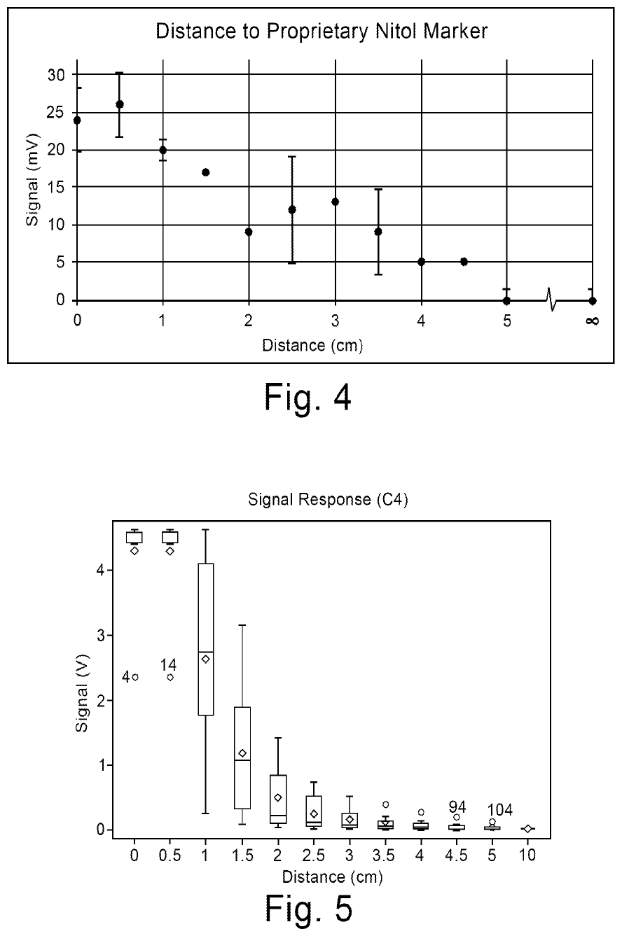

Preliminary benchtop verification was achieved through an assessment of detection range for a proprietary nitinol marker in an open-air environment. Signal voltage was recorded first with a negative control (∞ cm) and then at 0.5 cm increments from 5.0 cm to the tip of the probe (0 cm). The results of this study are plotted in FIG. 4. A positive return signal was expressed as a relative voltage increase from baseline. The strength of signal response generally attenuated inversely with distance, achieving a maximum amplitude of 24±4.2 mV at 0.5 cm. While a detection signal of 5 mV was registered at a proximity of 4.5 cm, error due to baseline drift could cause doubt that reliable detection could be attained at this distance.

Further discrepancies in the expected distance-to-signal relationship (i.e. between 2 and 3 cm) are likely attributable to fluctuations in the baseline signal. The data presented in FIG. 4 was collected on an oscilloscope and visualized by post processing.

Preliminary ex vivo validation was achieved through an assessment of detection range for a proprietary nitinol marker, the Somatex Tumark Vision. A collaborating surgical oncologist was able to identify the location of a marker placed beneath a mastectomy sample using the detector probe and an oscilloscope. The purpose of this qualitative study was to ensure that the presence of tissue did not hinder clip detectability. A successful trial was defined by the surgeon's ability to localize the marker by extrapolating a focal region of high signal strength.

In order to determine the optimal ratio of PLGA:IONP, five formulations were produced at percent weight ratios of 70:30 (C1), 60:40 (C2), 40:60 (C3), 30:70 (C4), and 20:80 (C5). The two constituents were added such that the total weight of each formulation was approximately 2.00 g. First, nanoparticles were introduced into the polymer through solvent casting. This involved dissolving PLGA, sourced from Akina, Inc., in a minimal volume of acetone for about 1 hour while stirring vigorously. When the PLGA was fully dissolved, a complementary amount of ION, sourced from Sigma-Aldrich, was added and stirred to encourage homogeneity. The PLGA-IONP solution was allowed to dry under vacuum for 24 hours after which the dried product was collected and weighed. High yields (91.4% to 95.3%) were achieved for each of the five formulations. At this stage, the consistency of the five compositions fell along a continuum of rubbery to flaky in accordance with proportion of PLGA such that PLGA-rich formulations were rubbery.

Compaction and shaping of the markers were achieved through compression molding. First, samples of 0.50 g were isolated and heated to approximately 80° C. to bring the PLGA to its glass-transition temperature and to evaporate off any residual acetone. Once cooled, the samples transitioned from a rubbery amorphous to a glassy crystalline state. These glassy samples were fragmented and collected in a stainless-steel die into which they could be compressed. A smooth rod of equal diameter was forced into the die under pressure supplied by a vice and moderate heat (40° C.) supplied by a heat gun.

The resultant composites were compact, crystalline phase discs approximately 0.50 g in weight and measuring about 8 mm in diameter and 3 mm in depth. Samples which readily transitioned to a glassy state proved much easier to process and compact. This favored formulations that were high in PLGA content, as the polymer was able to act as a binder. As such, the formulation containing only 20 wt % PLGA did not contain sufficient PLGA content to readily compact and maintain geometry. Rather, the compaction product remained powdery and non-cohesive. Thus, no marker prototype could be produced from this formulation.

Detection distance validation of the comprehensive intraoperative localization system, comprised of both the composite biopsy marker and detector, was achieved through an open-air benchtop proximity study. The detector probe was isolated from transient metal objects and the system was powered on, and the LabVIEW GUI was initiated. Prototype samples from each of the four compositions (C1, C2, C3, and C4) were placed 10 cm away from the distal end of the probe surface and a signal baseline was set. Signal voltages were recorded in 0.5 cm increments from 5.0 cm to the tip of the probe (0 cm). The marker prototype was held at each data collection interval for approximately 1 second. This process was repeated over 8 to 10 trials for each of the four tested compositions. It is preferable that the measurement intervals are positioned orthogonal to the probe tip for consistency. This is a consequence of arrangement of the coupled coils within the probe's distal end. The orientation of the coils produces a field that projects at greater strength in the orthogonal direction. The coil is preferably arranged such that magnetic field strength is maximized in the axial direction.

Data collected through use of the LabVIEW user interface were programmatically exported to a spreadsheet for analysis. This information was used to evaluate various performance characteristics of the composite markers and detector system. Table 1 shows a collection of Least Square Mean (LSM) signal estimates for each composition at a given distance. Each prototype formulation was detectable (a signal differential of about 10 mV) from 3.0 to 3.5 cm away. In Table 1 the signal estimates are at alpha=0.05 for each composite at each recorded distance.

TABLE 1

| Distance | Signal Estimate (V) | | | |
|---|---|---|---|---|
| (cm) | C1 | C2 | C3 | C4 |
| 0.0 | 4.667 | 4.914 | 4.726 | 4.296 |
| 0.5 | 3.296 | 4.372 | 3.837 | 4.295 |
| 1.0 | 1.001 | 1.523 | 1.514 | 2.621 |
| 1.5 | 0.138 | 0.151 | 0.097 | 1.167 |
| 2.0 | 0.060 | 0.079 | 0.042 | 0.491 |
| 2.5 | 0.025 | 0.033 | 0.022 | 0.236 |
| 3.0 | 0.013 | 0.027 | 0.016 | 0.145 |
| 3.5 | 0.009 | 0.020 | 0.013 | 0.089 |
| 4.0 | 0.005 | 0.014 | 0.012 | 0.059 |
| 4.5 | 0.004 | 0.011 | 0.010 | 0.038 |
| 5.0 | 0.003 | 0.008 | 0.006 | 0.018 |

To determine optimal marker composition as it relates to detectability the various formulations were compared to one another. Compositions C4, containing the greatest proportion of IONP among the samples tested, yielded the greatest signal response at each distance. A Tukey's HSD Test was performed to take the differences of the means of each composition and to test this hypothesis. The results of this test are collected in Table 2 wherein provided is Tukey's HSD Test to compare the means of each composition. At very close range, each marker composition was capable of saturating the field and forcing the circuits comparator to output maximum voltage. Differences among the markers are likely attributed to differences in relative baselines. This could be the result of a heterogenous distribution during solvent casting or compression molding. Uneven allocation of IONP among the workpieces of a given formulation would leave some variation among the samples from a given formulation. In each case, the absolute voltage signal achieved was 9.0 V. The benefit of added IONP content in C4 is advantageous at further distances such as greater than 1.0 cm. This is likely due to the sensitive relationship between distance and signal response in this regime. The IONP content of C4 is sufficient to cause the circuit's comparator to begin switching at a greater distance than the other compositions.

TABLE 2

| Distance (cm) | C4-C1 | C4-C2 | C4-C3 |
|---|---|---|---|
| 0 | −0.3708 | −0.6183* | −0.4302 |
| 0.5 | 0.9993 | −0.0765 | 0.4583 |
| 1.0 | 1.6200 | 1.0981 | 1.1066 |
| 1.5 | 1.0288* | 1.0154* | 1.0693* |
| 2.0 | 0.4303* | 0.4117* | 0.4486* |
| 2.5 | 0.21102* | 0.19817* | 0.21474* |
| 3.0 | 0.13203* | 0.11832 | 0.12979* |
| 3.5 | 0.08053* | 0.06940 | 0.07564 |
| 4.0 | 0.05328* | 0.04417 | 0.04646 |
| 4.5 | 0.03311 | 0.02669 | 0.02790 |
| 5.0 | 0.015181 | 0.010513 | 0.012068 |
| 10.0 | 0 | 0 | 0 |

* indicates significance at a p-value of 0.05 or less

In addition to the Tukey's Test, a Fischer's Least Significance Difference (LSD) Test was performed to compare C4 to C1, C2, and C3. The LSD Test is less conservative than Tukey' Test, and it is thus not surprising that it yielded greater significances among the compositions. Table 3 provides p-values from the LSD test and confirms that C4 produces a significantly stronger response signal compared to the other three prototype formulations.

TABLE 3

| Distance (cm) | C1 | C2 | C3 |
|---|---|---|---|
| 0 | 0.0326* | 0.0013 | 0.0155* |
| 0.5 | 0.1714 | 0.9832 | 0.5082 |
| 1.0 | 0.0347* | 0.0709 | 0.0695 |
| 1.5 | 0.0001** | 0.0001** | <0.0001** |
| 2.0 | 0.0015** | 0.0014** | 0.0007** |
| 2.5 | 0.0021** | 0.0025** | 0.0013** |
| 3.0 | 0.0040** | 0.0059** | 0.0032 |
| 3.5 | 0.0088** | 0.0146* | 0.0092** |
| 4.0 | 0.0104* | 0.0209* | 0.0164* |
| 4.5 | 0.0236* | 0.0457* | 0.0386* |
| 5.0 | 0.0892 | 0.2024 | 0.1527 |
| 10.0 | — | — | — |

*indicates significance at a p-value of 0.05 or less
**indicates significance at a p-value of 0.01 or less

A final aspect of detectability relates to the capacity to reliably distinguish one distance from the next. If a user interface is to be created which linearizes distance-to-marker signal, it is essential that this signal predictably attenuate with distance. Reliable signal discrimination related to distance was analyzed by comparing the distribution of signal responses to C4 for each distance among 10 trials. The results are provided in FIG. 5 wherein provided is a graphical representation of the distribution of signal responses to C4 at each distance among the 10 trials. Beginning at about 1 cm, the distribution of signal responses for each distance are relatively distinct. Though some overlap between adjacent distances (1.5 cm and 2.0 cm) does exist, this is partly due to the arbitrarily defined recording intervals. If increments were defined at 1 cm intervals rather than 0.5 cm intervals, very little overlap would exist. The data illustrates that signal response is indicative of the composite marker's location within about 0.5 cm.

The invention has been described with reference to the preferred embodiments without limit thereto. One of skill in the art would realize additional embodiments and improvements which are not specifically stated but which are within the meets and bounds of the claims appended hereto.

Claimed is:

1. A breast tissue marker and localization system comprising:
a biocompatible marker comprising a polymeric binder and a magnetically susceptible material wherein said biocompatible marker is suitable for implantation for localization of an area of pathological interest; and
a detector capable of detecting a response magnetic field in said biocompatible marker.

2. The breast tissue marker and localization system of claim 1 wherein said polymeric binder is selected from the group consisting of poly(lactic-co-glycolic acid); polyethylene glycol, poly(vinylpyrrolidone), poly(vinyl alcohol) and chitosan.

3. The breast tissue marker and localization system of claim 1 wherein said marker comprises at least 20 wt % of said polymeric binder and no more than 80 wt % of an iron oxide nanoparticle.

4. The breast tissue marker and localization system of claim 1 wherein said magnetically susceptible material is an iron oxide nanoparticle.

5. The breast tissue marker and localization system of claim 4 wherein said iron oxide nanoparticle is Fe₃O₄.

6. The breast tissue marker and localization system of claim 1 wherein said magnetically susceptible material is an iron (III) oxide nanoparticle.

7. The breast tissue marker and localization system of claim 6 wherein said iron (III) oxide nanoparticle is Fe₂O₃.

8. The breast tissue marker and localization system of claim 1 wherein said magnetically susceptible material has a particle size of 1 to 150 nm.

9. The breast tissue marker and localization system of claim 8 wherein said magnetically susceptible material has a particle size of 1 to 10 nm.

10. The breast tissue marker and localization system of claim 1 further comprising a surface treatment on said magnetically susceptible material.

11. The breast tissue marker and localization system of claim 10 wherein said surface treatment comprises silica or dextran.

12. The breast tissue marker and localization system of claim 1 further comprising an iron chelator in said polymeric binder.

13. The breast tissue marker and localization system of claim 1 wherein said biocompatible marker further comprises a surface coating.

14. The breast tissue marker and localization system of claim 13 wherein said surface coating comprises fibronectin.

15. The breast tissue marker and localization system of claim 1 further comprising a shielding device.

16. The breast tissue marker and localization system of claim 15 wherein said shielding device comprises a cylinder on a probe tip of said detector.

17. The breast tissue marker and localization system of claim 15 wherein said shielding device comprises a mu metal.

18. The breast tissue marker and localization system of claim 1 wherein said detector is capable of detecting said biocompatible marker at a distance of up to 10 cm.

19. The breast tissue marker and localization system of claim 18 wherein said detector is capable of detecting said biocompatible marker at a distance of up to 5 cm.

20. The breast tissue marker and localization system of claim 19 wherein said detector is capable of detecting said biocompatible marker at a distance of up to 4.5 cm.

21. The breast tissue marker and localization system of claim 20 wherein said detector is capable of detecting said biocompatible marker at a distance of up to 3 cm.

22. The breast tissue marker and localization system of claim 1 further comprising a graphic user interface capable of reporting a distance between said detector and said biocompatible marker.

23. The breast tissue marker and localization system of claim 22 wherein said reporting is selected from audible and optical.

24. The breast tissue marker and localization system of claim 1 wherein said detector is a handheld detector.

25. The breast tissue marker and localization system of claim 1 wherein said response magnetic field is induced by said detector.

26. A method for localizing an area of pathological interest comprising:
placing a biocompatible marker in a position relative to said area of pathological interest wherein said biocompatible marker comprises a polymeric binder and a magnetically susceptible material wherein said biocompatible marker is suitable for implantation for localization of an area of pathological interest; and
detecting said biocompatible marker by a detector capable of detecting a response magnetic field in said biocompatible marker.

27. The method for localizing an area of pathological interest of claim 26 wherein said detecting is intraoperative.

28. The method for localizing an area of pathological interest of claim 26 wherein said placing comprises injection.

29. The method for localizing an area of pathological interest of claim 26 comprising placing multiple biocompatible markers in multiple positions.

30. The method for localizing an area of pathological interest of claim 26 comprising developing a detection baseline prior to said detecting.

31. The method for localizing an area of pathological interest of claim 26 wherein said polymeric binder is selected from the group consisting of poly(lactic-co-glycolic acid); polyethylene glycol, poly(vinylpyrrolidone), poly(vinyl alcohol) and chitosan.

32. The method for localizing an area of pathological interest of claim 26 wherein said marker comprises at least 20 wt % of said polymeric binder and no more than 80 wt % of an iron oxide nanoparticle.

33. The method for localizing an area of pathological interest of claim 26 wherein said magnetically susceptible material is an iron oxide nanoparticle.

34. The method for localizing an area of pathological interest of claim 33 wherein said iron oxide nanoparticle is Fe₃O₄.

35. The method for localizing an area of pathological interest of claim 26 wherein said magnetically susceptible material is an iron (III) oxide nanoparticle.

36. The method for localizing an area of pathological interest of claim 35 wherein said iron (III) oxide nanoparticle is Fe₂O₃.

37. The method for localizing an area of pathological interest of claim 26 wherein said magnetically susceptible material has a particle size of 1 to 150 nm.

38. The method for localizing an area of pathological interest of claim 37 wherein said magnetically susceptible material has a particle size of 1 to 10 nm.

39. The method for localizing an area of pathological interest of claim 26 further wherein said magnetically susceptible material comprising a surface treatment.

40. The method for localizing an area of pathological interest of claim 39 wherein said surface treatment comprises silica or dextran.

41. The method for localizing an area of pathological interest of claim 26 further comprising an iron chelator in said polymeric binder.

42. The method for localizing an area of pathological interest of claim 26 wherein said biocompatible marker further comprises a surface coating.

43. The method for localizing an area of pathological interest of claim 42 wherein said surface coating comprises fibronectin.

44. The method for localizing an area of pathological interest of claim 26 further comprising a shielding device.

45. The method for localizing an area of pathological interest of claim 44 wherein said shielding device comprises a cylinder on a probe tip of said detector.

46. The method for localizing an area of pathological interest of claim 44 wherein said shielding device comprises a mu metal.

47. The method for localizing an area of pathological interest of claim 26 wherein said detector is capable of detecting said biocompatible marker at a distance of up to 10 cm.

48. The method for localizing an area of pathological interest of claim 47 wherein said detector is capable of detecting said biocompatible marker at a distance of up to 5 cm.

49. The method for localizing an area of pathological interest of claim 48 wherein said detector is capable of detecting said biocompatible marker at a distance of up to 4.5 cm.

50. The method for localizing an area of pathological interest of claim 49 wherein said detector is capable of detecting said biocompatible marker at a distance of up to 3 cm.

51. The method for localizing an area of pathological interest of claim 26 further comprising a graphic user interface capable of reporting a distance between said detector and said biocompatible marker.

52. The method for localizing an area of pathological interest of claim 51 wherein said reporting is selected from audible and optical.

53. The method for localizing an area of pathological interest of claim 26 wherein said detector is a handheld detector.

54. The method for localizing an area of pathological interest of claim 26 wherein said response magnetic field is induced by said detector.
